(19) United States
(12) Patent Application Publication
(10) Pub. No.: US 2012/0109031 A1
(43) Pub. Date: May 3, 2012

(54) SUPPORT BANDAGE

(75) Inventors: Matthias Vollbrecht, Herzberg am Harz (DE); Holger Reinhardt, Kempen (DE); Gert-Peter Bruegemann, Koln (DE); Andreas Goesele-Koppenburg, Loerrach (DE); Raymond Best, Stuttgart (DE); Andree Ellermann, Ettlingen (DE); Alfio Albasini, Riazzino (CH); Christian Liebau, Braunschweig (DE)

(73) Assignee: OTTO BOCK HEALTHCARE GMBH, Duderstadt (DE)

(21) Appl. No.: 13/378,821

(22) PCT Filed: Jun. 16, 2010
(86) PCT No.: PCT/EP2010/003624

§ 371 Date: Dec. 16, 2011

(30) Foreign Application Priority Data
Jun. 16, 2009 (DE) 10 2009 025 415.3
Oct. 22, 2009 (DE) 10 2009 050 383.8

(51) Int. Cl. A61F 5/01

(52) U.S. Cl. 602/5

(57) ABSTRACT

The invention relates to a support bandage (1) comprising a base body (2) that is made of an elastic textile material and that has an inner side facing the body of a bandage user and an outer side facing away from the body of the bandage user. An anti-slip coating (5) is arranged on the inner side. The support bandage (1) has at least one supporting strap (10) that is reversibly arranged on the outer side of the base (2) by way of form-fitting elements (21, 16).

The invention relates to a support bandage comprising a base body that is made of an elastic textile material and that has an inner side facing toward the body of a bandage user and an outer side facing away from the body of the bandage user, with an anti-slip coating arranged on the inner side.

Such a support bandage serves to support a joint or a limb, for example a thigh or lower leg. Support bandages can be applied to the ankle, to the knee, and to the joints of the upper extremities, and it is likewise possible for such support bandages to be arranged along muscle strands without bridging any joints. The support bandages are worn in the context of rehabilitation measures in order to promote the healing process after an injury and, if appropriate, to allow continuation of a sports activity. In addition, support bandages can be used to alleviate pain in degenerative diseases. A further possible use of support bandages is to provide increased blood flow in the muscle tissue by-applying a uniform pressure. The increased blood flow improves the supply of oxygen-rich blood to the muscles, as a result of which an improvement in performance can be achieved. The use of support bandages can improve proprioception. The receptors are stimulated by pressure from outside, which leads to improved coordination and to a greater level of activity of the muscles.

DE 40 91 302 T1 describes a support bandage with a main part made of an elastic material and a non-elastic material or only of an elastic material. Some of the elastic material is impregnated in order to modify the elasticity in some areas.

DE 3902434 A1 relates to a support bandage designed for limbs and/or joints and made of an elastic textile material, in which anti-slip elements made of silicone material are arranged on the inner side of the elastic textile material that comes into contact with the skin. The anti-slip elements can be designed as individual patches; they can be arranged on the inner side of the support bandage in almost any desired, configuration.

The object of the present invention is to make available an improved support bandage with which the supporting effect can be adjusted individually and can be adapted to the respective physical characteristics and to changes in the bandage or in the demands made on the bandage.

According to the invention, this object is achieved by a support bandage having the features of the main claim. Advantageous embodiments and refinements of the invention are set forth in the dependent claims.

The support bandage according to the invention, comprising a base body that is made of an elastic textile material and that has an inner side facing toward the body of a bandage user and an outer side facing away from the body of the bandage user, with an anti-slip coating arranged on the inner side, is characterized in that the support bandage has at least one supporting strap, which is arranged reversibly on the outer side of the base body by way of form-fitting elements, and the supporting strap can have at least one flexible, non-elastic section. By arranging a supporting strap on the outer side of the base body, it is possible to individually apply an increased elastic restoring force to those areas that require increased compression. It is thus possible to use the support bandage in an extremely versatile manner and, for different indications, to adapt it individually to the respective patient by means of a different arrangement of the supporting strap or of a plurality of supporting straps on the outer side of the support bandage. By means of the supporting strap being arranged reversibly on the outer side via form-fitting elements, it is possible to individually adjust the supporting strap and, after the support bandage has been fitted in place, to vary the arrangement of the strap relative to the base body, for example in order to maintain the original tension if the restoring force decreases as a result of warming of the bandage or fatigue of the elastic elements inside the base body. The form-fitting elements themselves can be arranged reversibly on the outer side of the base body, such that areas particularly suitable for fastening the supporting straps can be fastened individually. In addition to reversible fastening of the form-fitting elements or areas of form-fitting elements, these can also be fastened and fixed individually, for example after the bandage has been adapted to the user by a therapist, who positions the form-fitting elements and then fixes them, for example by sewing, adhesive bonding or welding.

The supporting strap can consist of only a non-elastic section, so as to ensure fixed bearing and stiffening of the support bandage. The flexible, non-elastic supporting strap is fixed on the base body and stabilizes the area on which the base body bears, for example a joint. Non-elastic is to be understood as meaning that a macroscopic elastic deformation of the material of the supporting strap is not envisioned, the elasticity inherent to every material being accepted as unavoidable.

In an alternative, the supporting strap has an elastic section arranged parallel to the non-elastic section, and the non-elastic section is secured, on the elastic section. By way of the flexible, non-elastic section, it is possible to exert a high degree of compression on the base body and therefore on the user of the support bandage. The elastic section arranged parallel thereto makes it possible to introduce a certain degree of flexibility into the supporting strap, so as to be able to follow the elastic stretching of the base body. The non-elastic section arranged in parallel constitutes a stretch-limiting means, which can be used at the same time as a safety feature against overloading. The elasticity of the supporting straps can be chosen individually, such that different elasticities of the supporting straps can also be present on a bandage. The elasticity needed in each case is chosen according to the requirements of the bandage user or of a therapist, the elasticity being adjustable from zero, i.e. substantially non-elastic, to virtually infinity.

The non-elastic section can be fixed permanently on the elastic section and can have a length that is different than the length of the unstretched elastic section. Depending on the length ratios, it is thus possible to have a purely non-elastic strap that is applied to the base body and, for example, permits fixing of the compression by the base body. If the elastic effect of the supporting strap is then to be exploited, the non-elastic section can be released, for example by means of the non-elastic section being cut through. If the non-elastic section is longer than the unstretched elastic section, the non-elastic section acts as a length limiter and prevents the elastic section from being subjected to a force that is too great. In this way, the elastic section can be protected from damage.

If the non-elastic section is fastened reversibly on the elastic section, e.g. via form-fitting elements configured as hooks, eyelets, flaps or a velcro-type fastener, it is possible to use the above-described effects individually. Removal of the non-elastic sections results in a purely elastic configuration, since the non-elastic sections have been taken out of operation. If the external circumstances then change, for example in the course of a sports competition or in the event of decreasing compression of the base body and of the supporting strap, there is the possibility of strengthening or blocking the elastic section with the non-elastic section.

The elastic and non-elastic sections are preferably arranged over each other and can at least partially cover each other. For example, the elastic section can be wider than the non-elastic section and receive the latter.

It is possible that several elastic and non-elastic sections arranged parallel to one another are arranged in series, such that a length graduation is readily possible. It is likewise possible to adjust the overall extension by means of individual non-elastic sections being taken out of operation, such that the elastic sections respectively arranged parallel thereto are easily stretchable.

A non-elastic strap segment can be arranged between two elastic sections, such that the supporting strap does not consist entirely of an elastic base section, although that is also possible. The supporting strap then consists of an elastic base section, on which a non-elastic section is arranged.

If provision is made that several elastic and non-elastic sections arranged parallel to one another are arranged in series, the individual elasticities can be coupled with one another, thereby affording a wide range of variation for adjusting the functionality of the strap. For example, if the non-elastic strap is shorter than the unstretched elastic strap, the elasticity can be increased in steps by cutting through several non-elastic sections. In the case of reversible fastening, greater elastic extensibility can be made available by undoing the connection, and this extensibility can be limited to the desired extent by once again fastening the non-elastic section.

As long as the non-elastic section is shorter than the unstretched elastic section, the supporting and fixing strap can preferably be used, as a stabilizing strap which, for example in the first phase of trauma therapy, is used to provide strong stabilization. In the first phase of the therapy, it may be necessary to support and immobilize the joint or the limb. After a certain period of immobilization, the quickest possible return to mobility of the respective limb is indicated in the context of early functional therapy. For this purpose, the short non-elastic section can then be cut through as soon as the barrier, formed for the elastic section by the non-elastic section, is canceled. The elastic section is now effective and can permit a resiliency of the supporting and fixing strap.

By contrast, if the non-elastic section is longer than the unstretched elastic section, the elastic section initially acts as a force-transferring means. When the maximum, extension limit of the elastic section is reached, the non-elastic section serves as a safety against overloading. For this purpose, provision is in particular made that the non-elastic section, although longer than the unstretched elastic section, is shorter than the maximum length of the elastic section, such that the non-elastic section becomes effective before the maximum extension of the elastic section is reached.

In a development of the invention, several elastic sections are arranged parallel to one another, for example two or more elastic strap sections over one another, in order to increase the spring force of the elastic sections. If the elasticity at which a greater extension is reached at a constant force is then to be increased, it is necessary merely to cut through or remove one or more elastic sections in order to achieve an individual adjustment of the elasticity and functionality of the strap.

The elastic and non-elastic sections preferably have the same width, so as to make available a strap with continuously the same width. For this purpose, it is necessary, where appropriate, that the non-elastic strap segment or the non-elastic strap segments also have the same width as the sections. Alternatively, provision is made that the non-elastic and elastic sections have different widths, the non-elastic section being preferably narrower than the elastic section and being arranged thereon.

The fixing means for the supporting strap can be designed as hook and loop areas, which fixing means can be arranged on the non-elastic sections or on the non-elastic strap segments. Even when the elastic and non-elastic sections are not connected permanently to each other, they can have different lengths. It is likewise possible that several elastic sections are arranged parallel to one another, such that the extension force to be applied can be varied by an accumulation of the elastic sections.

The base body is preferably designed as a sleeve, so as to be pulled over the intended joint or the intended limb. Alternatively, the base body can also be flat, and provided with connecting elements for closing the base body around the joint or the limb. The connecting elements are, for example, velcro-type fasteners, eyelets, hooks, clasps or the like.

The anti-slip coating is advantageously arranged only in some areas on the inner side of the base body, so as to be able to permit correct assignment of the base body to the limb of the bandage user. If the coating covers the entire surface area, there is the danger that the necessary permeability to moisture will not be provided. A coating provided in some areas is generally sufficient for arranging the support bandage securely on the skin surface and avoiding inadvertent twisting or shifting of the bandage on the skin surface. It is possible and envisioned to apply an air-permeable coating across the entire surface area.

Areas with form-fitting elements for fastening the supporting strap can be arranged on the outer side of the base body, which form-fitting elements can be designed as hook or loop areas of a velcro-type fastener. It is likewise possible that other form-fitting elements are present, for example tabs, flaps, eyelets, press buttons or the like. The areas are preferably arranged at physiologically expedient locations, such that a corresponding shifting of the supporting strap is made easier after the bandage has been applied. The areas with the form-fitting elements can be arranged reversibly on the outer side of the base body, such that they can be adapted individually.

The outer side of the base body can be designed as a loop-covered layer, and this loop-covered layer can completely cover the outer side of the base body. The form-fitting elements of the supporting strap can be designed as hook elements, such that the supporting strap can be fastened at any desired location on the outer side of the base body. Alternatively, the outer side of the base body can also be covered completely with hook elements, such that the loop-covered areas of the supporting strap can adhere to the assigned locations. The base body can have at least one recess in order to make available an area with a reduced pressure effect, for example so as not to exert a pressure force on the kneecap or the elbow. A body part, for example the thumb in the case of a wrist bandage, can also be pushed through the recess in order to avoid slipping.

The supporting strap can have a force indicator in order to provide the user of the support bandage with feedback and information on the extent of the force presently applied by the supporting strap and thus exerted on the base body. The force indicator can be designed as an element moving in a window, for example on a window in the non-elastic section. Likewise, the extension force presently applied can be indicated by a change of color or by the shifting of a marking. If the base body becomes slack, the force indicator makes it possible to provide the bandage user with information to the effect that the supporting strap needs to be retightened. The base body can have a guide in which the supporting strap can be inserted in order to permit a movable bearing inside the base body, without the fixing effect being adversely affected thereby. This guide can be designed as a flap, slit or the like.

In addition to a preferred rectilinear design of the supporting strap, it is possible for the latter to be designed in a curved basic shape, in order to permit better adaptation to the physiological conditions of the bandage user. For example, an S-shaped design of the supporting strap is favorable for applying the force around the patella.

Elastic elements can be secured, on the textile material or the supporting strap to modify the elasticity of the support bandage in some areas, for example by means of several elastic elements being coupled to one another or individual elastic elements being fastened on specific areas of the textile material in order to apply tension. The textile material can be elastic. The elasticities of the base body or of the supporting strap can also be adjusted by way of perforations, which are formed in the respective material.

The material of the base body preferably possesses shear stability, such that the top face cannot be shifted relative to the underside facing toward the body. The shear forces act parallel to the plane of the base body. The shear stability is influenced by the structure of the material and the thickness of the material. The thicker the material, the lower the shear stability, since the top face can be moved more easily relative to the underside.

The supporting straps can have different tensile elasticities, which can be generated through different materials, perforations or production methods.

In another embodiment, the supporting strap can be arranged at least partially on the inner side of the base body, such that the supporting strap at least partially bears directly on the skin surface and causes a stimulation of the underlying muscle. The positional stability is ensured, for example, by an anti-slip coating provided on the inner side of the supporting strap facing toward the skin surface, which is assisted by the positional stability of the base body on the limb.

Knobs, press elements, projections, ball segments or other devices can be mounted on the supporting strap which, locally increase the pressure on the affected muscle area or the affected part of the limb. It is possible to provide individual devices or elements and likewise several devices or elements, in particular in order to increase proprioception. Alternatively or in addition, the devices for locally increasing the pressure can be arranged on the base body, where they act on the intended location. An increase in the pressure can take place by applying and tightening the supporting strap, which is placed over the device and fastened on the base body. The devices for locally increasing the pressure can be arranged on the outer side of the base body, and it is likewise possible that the devices for locally increasing the pressure are arranged on the inner side of the supporting strap and/or of the base body. When arranged on the inner side, direct contact with the skin surface can be produced, and likewise good screening and coverage by the base body and if appropriate by the supporting strap. An arrangement on the outer side of the base body facilitates positioning at the correct location. The devices for locally increasing the pressure can be fastened reversibly on the base body and/or the supporting strap, for example by way of a velcro-type fastener. The devices are, for example, made of plastic and are preferably of rounded shape, in order to ensure a comfortable wear.

The base body can have passages for the supporting strap, such that the ends or also intermediate areas of the supporting strap can be fastened on the top face of the base body. In this way, the form-fitting elements via which the supporting strap can be fastened on the base body can remain easily accessible on the outer side of the support bandage, while parts of the supporting strap on the inner side are in direct contact with the skin surface. It is likewise possible that the entire supporting strap is arranged on the inner side and is covered by the base body when the support bandage is fitted. The support bandage is intended for use as a bandage supporting the muscles, and it is not intended to stabilize a joint by limiting the movement by diversion of forces to outer stiffening elements. Rather, a muscle-supporting function is to be provided by exerting pressure on the muscle lying underneath the supporting strap. For this purpose, the supporting strap is fastened on the base body substantially along the muscle course, that is to say along the muscle or at an acute angle diagonally to the longitudinal extent of the muscle. By means of the reversible arrangement of the supporting strap on the base body, repeated use is possible, in particular an adjustment and an increase or reduction of the tension on the muscle is possible, during use, by simply shifting the ends of the supporting strap on the base body. The supporting straps thus act as compression elements, which act on the respective muscle, and not as securing elements which, by circular tension, are intended to shift an orthosis on the limb or limit a movement. Rather, partial pressure is exerted on the muscle without obstructing the blood supply by application of elastic compression straps. Both the base body and also the compression straps, that is to say all the components of the bandage, are flexible, such that no joint stiffening or movement obstruction takes place should a bandage be fitted over a joint. Provision is also made that the bandage is only worn such that no joint is covered. The extent and course of the locally acting compression are individually adjustable and can be readjusted as often as necessary.

Illustrative embodiments of the invention are explained in more detail below with reference to the attached figures, in which:

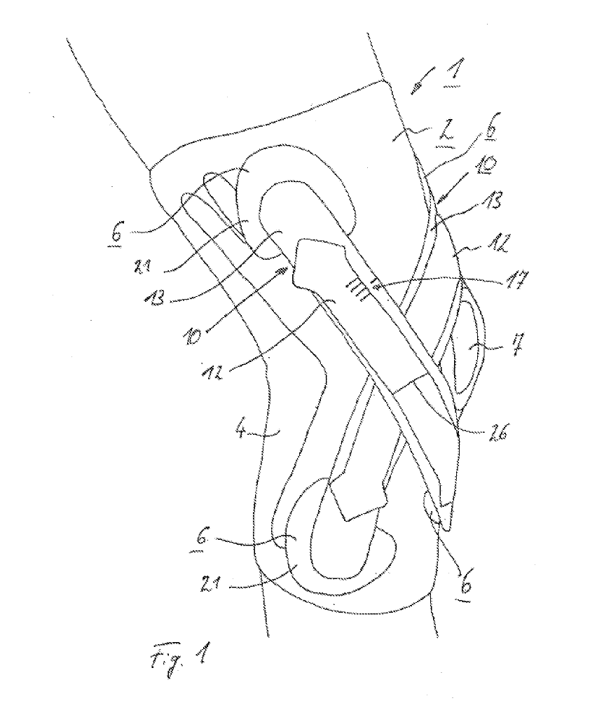

FIG. 1 shows a first embodiment of the support bandage as a knee bandage;

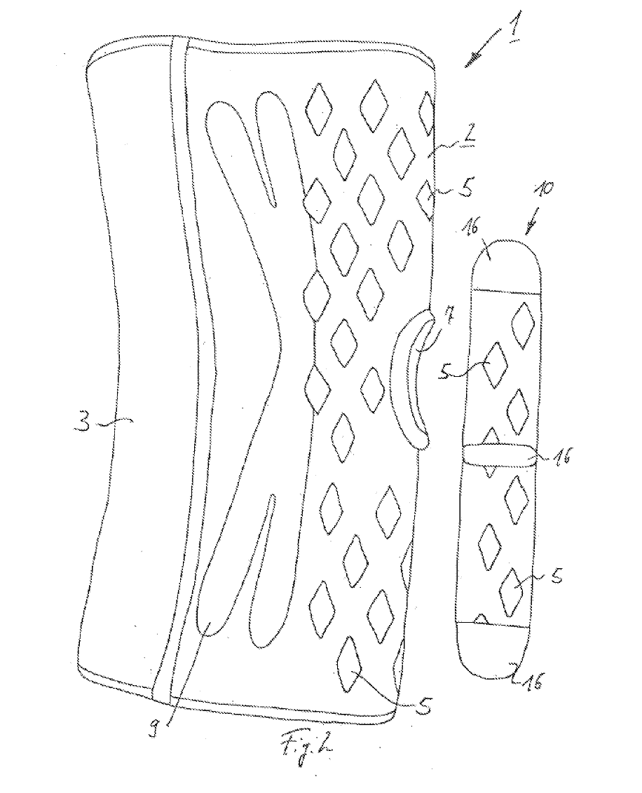

FIG. 2 shows an inner side of the knee bandage according to FIG. 1;

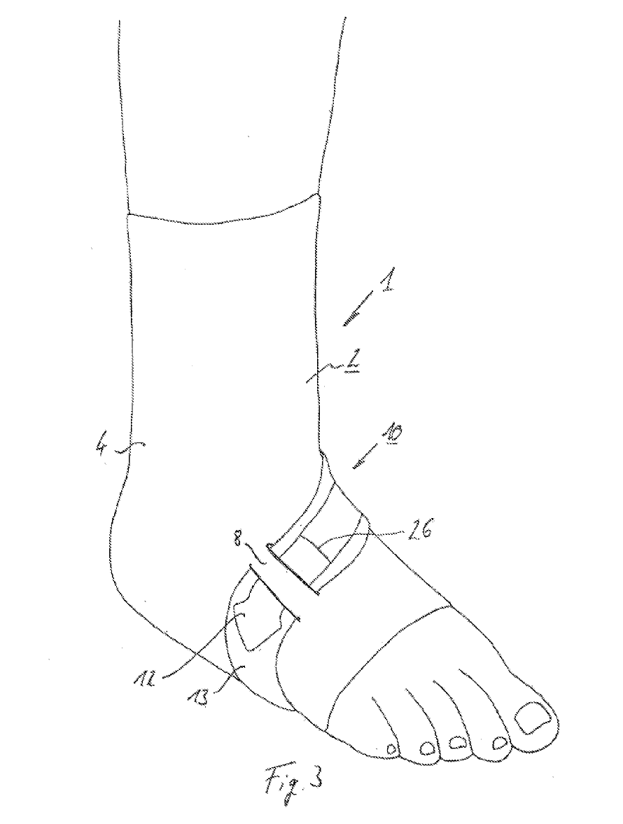

FIG. 3 shows a support bandage as an ankle bandage;

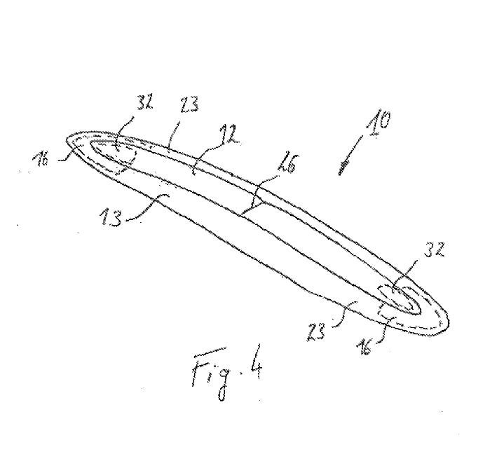

FIG. 4 shows a supporting strap on its own;

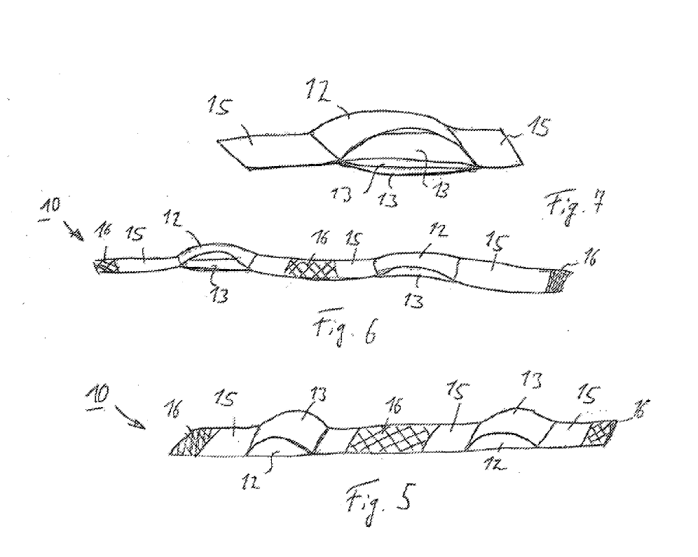

FIG. 5 shows a variant of the supporting strap with a short elastic section;

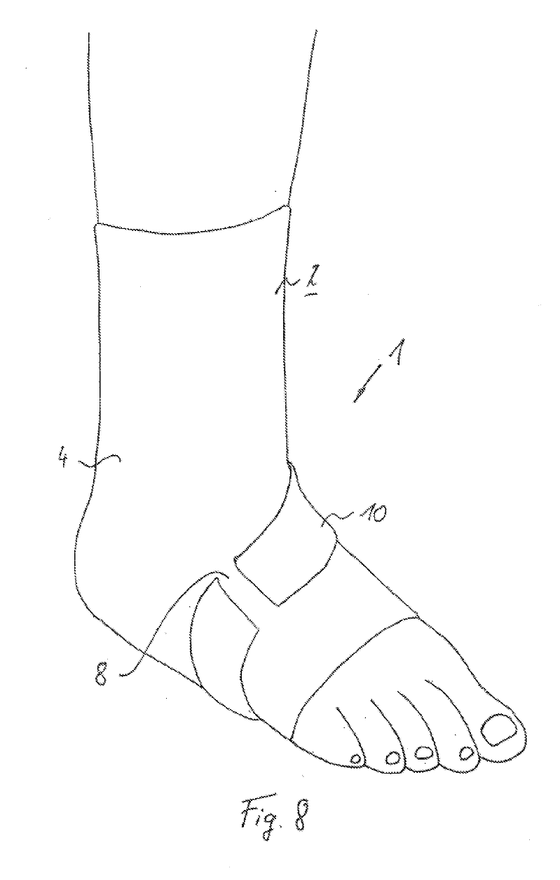

FIG. 6 shows a variant of the invention with a short non-elastic section;

FIG. 7 shows a detail with several elastic sections connected in parallel;

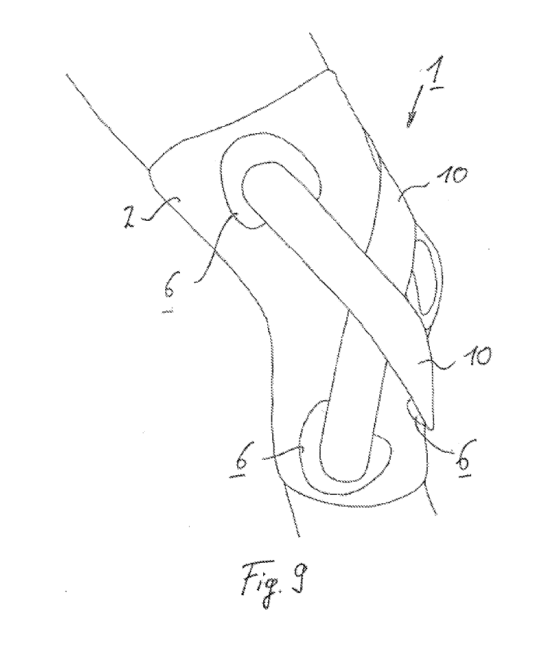

FIG. 8 shows a variant of FIG. 3;

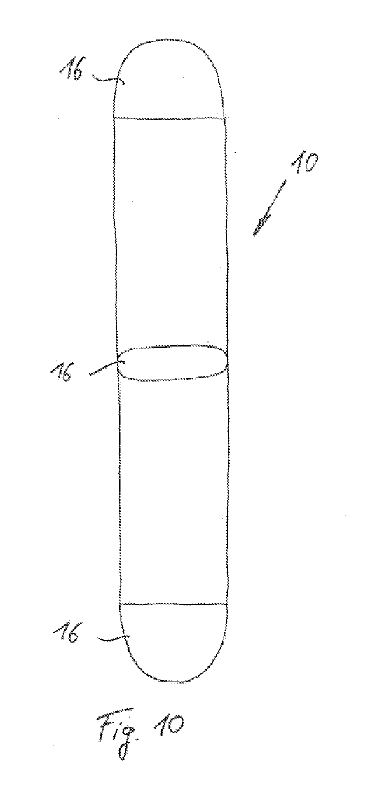

FIG. 9 shows a variant of FIG. 1;

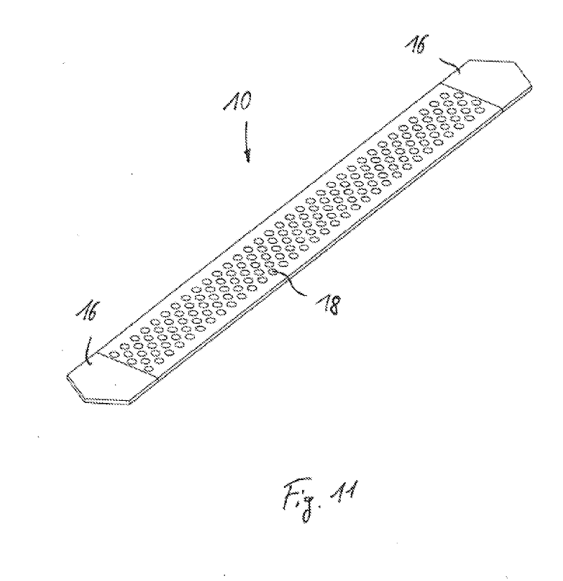

FIG. 10 shows a detail of a supporting strap;

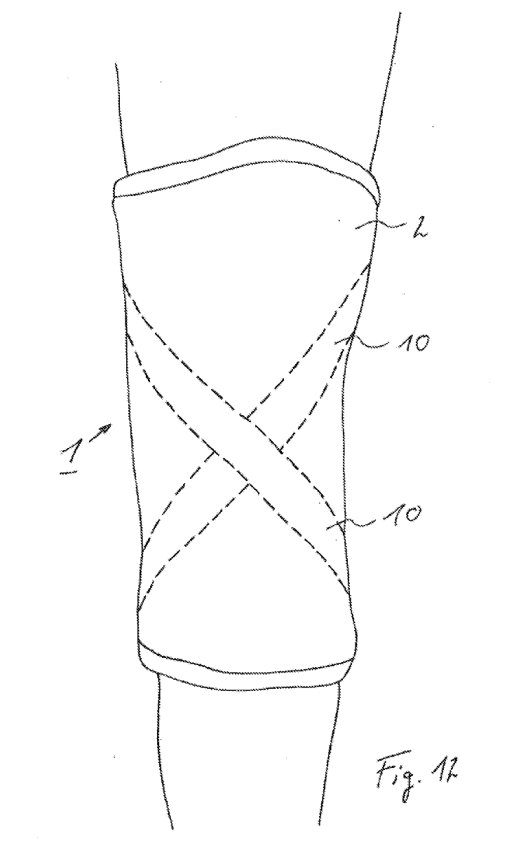

FIG. 11 shows a variant of the supporting strap;

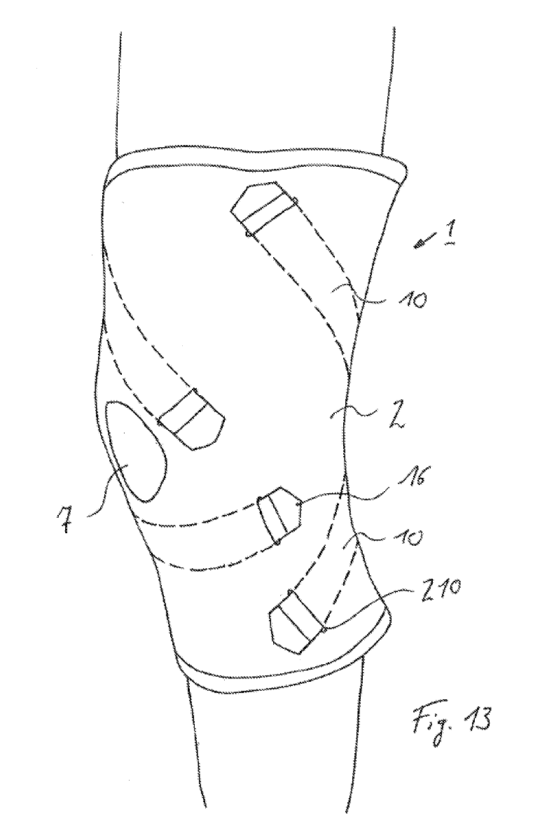

FIG. 12 shows a variant of the bandage with the supporting strap lying on the inside;

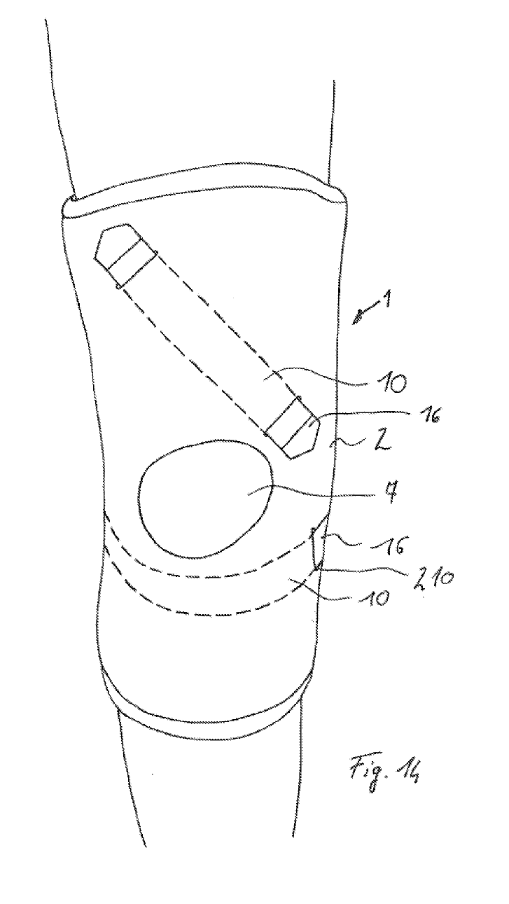

FIG. 13 shows an oblique plan view of FIG. 12; and

FIG. 14 shows a variant of FIG. 13.

A support bandage 1 with a base body 2 made of an elastic textile material is shown in FIG. 1. The support bandage 1, which is designed as a knee bandage, is shown in a state when fitted, and therefore only the outer side 4 is to be seen. A recess 7 is formed in the area of the kneecap in order to avoid undesired pressure on the kneecap. The outer side 4 of the base body 2 has separately marked areas 6 with form-fitting elements 21, for example in the form of hooks or loops of a velcro-type fastener. These areas 6 with form-fitting elements 21 can be arranged at places or areas that are deemed expedient from the physiological point of view, and the position and/or size of the areas 6 can vary depending on the indication. It is likewise possible that a large number of such areas 6 are arranged on the outer side 4 of the base body 2 and, for example, have a different color marking to permit correct assignment of the supporting strap 10, as will be described in more detail below. It is also possible and intended that the entire outer side 4 of the base body 2 is designed as a loop-covered layer, on which the areas 6 are visually delimited and marked.

Two supporting straps 10 are fastened on the outer side 4 of the base body 2 of the support bandage 1. They are fastened by way of form-fitting elements 16, which are explained in more detail later. The form-fitting elements 16 can, for example, be designed as hook elements via which, the ends of the supporting straps 10 are brought into contact with the areas 6 having the loop-covered layer 21 and can be fastened on the base body 2. In the embodiment shown, the supporting straps 10 are arranged crossing over each other on the outer side 4 of the base body 2, the point of intersection lying laterally alongside the patella. It is in this way possible to additionally support the knee joint and guide the patella.

The supporting straps 10 consist of an elastic section 13 and, arranged above this, a non-elastic flexible section 12. The non-elastic section 12 is secured via a fixing seam 26 on the elastic section 13, which can make up the entire length of the strap. To apply the supporting strap 10, the ends simply need to be placed in the areas 6 with the loop-covered layer 21. By virtue of the elasticity of the elastic section 13, it is possible to apply a tensile force, which acts between the securing areas 6 and, in the case of a knee bandage, leads to stability of the knee. By way of the flexible, non-elastic section 12, it is possible to apply, instead of an elastic tensile force, a more or less rigid compression for increasing the supporting effect.

Several line markings are arranged on the non-elastic section 12, making it possible to provide a. force indicator. The elastic section 13 likewise has a marking which, depending on the tensile force applied, lies opposite a corresponding marking on the non-elastic section 12. The farther away the marking on the elastic section 13 from the fixing seam 26, the greater the tensile force applied. Other force indicators 17 are envisioned and possible, for example windows, tabs, the arrangement of several markings on the elastic section 13 with a single reference marking on the non-elastic section 12, or similar.

In FIG. 2, the support bandage 1 according to FIG. 1 is shown with the inner side 3 facing outward. It will be noted that, on the inner side 3 of the support bandage 1, anti-slip coating points 5 are arranged on the base body 2 in order to avoid the support bandage 1 slipping on the skin surface when fitted. The anti-slip coating 5 is preferably made of a silicone material, although other materials, for example PU, can likewise be provided. In addition to the anti-slip coating 5 being arranged in points or patches, it is also possible to provide the anti-slip coating 5 over large surface areas.

It will also be seen in FIG. 2 that a stabilizing splint 9 for increasing the lateral stability is worked into the base body 2. The stabilizing splint 9 is made of a dimensionally stable material, preferably plastic, and permits a certain amount of flexibility in the medial and lateral directions.

FIG. 2 also snows the underside of the supporting strap 10. The supporting strap 10 can likewise have an anti-slip coating 5, which is advantageous if the supporting strap 10 is to be applied directly to the skin. It will also be seen that form-fitting elements 16 are arranged both on the upper and lower ends and also in the middle of the supporting strap 10. By way of these form-fitting elements 16, in particular hook areas, it is possible to secure the supporting strap 10 firmly and reversibly on the outer side 4 of the base body 2 of the support bandage 1.

FIG. 3 shows an ankle bandage 1 with a base body 2, in which a flap 8 serving as a guide for the supporting strap 10 is formed inside the base body 2. In addition to an integral design of the guide 8, the latter can also be sewn on. Other guide elements or means can likewise be provided in order to safely apply the supporting strap 10 to the bandage and therefore to the limb around which the support bandage 1 is fitted. Here too, elastic and non-elastic sections 13, 12 are arranged over each other. The non-elastic section 12 is fastened via the fixing seam 26 to the elastic section 13 arranged underneath the non-elastic section 12. By way of the position of the ends of the elastic section 13, it is possible to adjust the respectively applied tensile force and, therefore, the stabilizing effect of the supporting and fixing strap 10. By means of the supporting and fixing strap 10 being reversibly fastened, it is also possible to readjust it and to achieve a stepless adjustment of the forces applied to the support device 1. The arrangement of supporting and fixing straps 10 on the base body allows a highly differentiated force build-up in the support device. The supporting and fixing straps 10 increase the tension only in partial areas of the support bandage 1, such that forces can be applied to the limb in a deliberate manner. The by the elastic sections 13 is transferred via the anti-slip coating 5, which acts as an interface between the textile base body 2 and the skin. The different tensile forces or transverse forces are not obtained through a complicated technique of producing the base body 2, but can be achieved easily and on an individual basis without having to produce a specific support bandage 1. The form-fitting elements 21 on the outer side 4 of the support bandage 1 can be designed elastically as an interface, such that a wide range of use of the base body 2 is afforded. Predetermined areas or zones for fastening the supporting straps 1 on the outer side 4 of the support bandage 1 can be marked, in order to correctly support the joint. The tension can be readjusted by simply releasing, moving and then re-fastening the ends of the supporting strap 10.

FIG. 4 shows a schematic view of a fixing or supporting strap 10 comprising, in the embodiment shown, a continuous elastic strap section 13 which, on its top face 23, can be provided with, a loop-covered layer. The underside is provided with fixing means 16 for fastening the end areas to each other or to an orthopedic device, in particular the support bandage 1. These fixing means 16 can also be designed as hook or loop areas of a velcro-type fastener. Alternative fixing means 16, for example hooks, press buttons, eyelets or the like, can likewise be provided.

Arranged on the top face 23 of the elastic section 13 there is a non-elastic section 12 which, in the illustrative embodiment shown, is designed as an elongate strap element. The non-elastic section 12 is fixed on the elastic section 13 via a seam. 26 which, in the present illustrative embodiment, is arranged centrally. Other securing means can likewise be provided, for example the non-elastic section 12 can be welded or adhesively bonded on the elastic section 13. In principle, reversible fixing by means of velcro-type fasteners or the like is also possible.

Form-fitting elements 32 configured as hook areas of a velcro-type fastener are provided on the underside of the non-elastic section 12, that is to say on the side directed toward the top face 23 of the elastic section 13. By way of these form-fitting elements 32, it is possible to secure the non-elastic section 12 at almost any desired position between the maximum extent of the non-elastic section 12 and the securing seam 26. Here too, the non-elastic section 12 and the elastic section 13 of the strap 10 are arranged parallel to each other. By means of the non-elastic section 12 being fastened reversibly on the elastic section 13 by way of the velcro-type fasteners 32, it is possible to install an extension limit at a variable distance from a possibly permanent fixing point 26. The farther away from the securing point 2 6 that the respective end of the non-elastic section 12 is secured on the elastic section 13, the smaller the possible stretching of the elastic section 13. If the securing point is chosen such that the elastic section 13 in the unstretched state, i.e. without elastic deformation caused by application of a force, is longer than the non-elastic section 12, the area bridged by the non-elastic section 12 is robbed of its elastic effect. If the non-elastic section 12 then extends from one outer end to the other outer end of the strap 10, there is no longer any elasticity, and an elastic component is then restored only when the connection to the form-fitting elements 32 is released.

FIG. 5 shows a supporting or fixing strap 10 of elongate design. The supporting or fixing strap 1 has a uniform width and is shown lying flat in FIG. 5, At its ends, the supporting or fixing strap 10 has fixing means 16 with which the strap ends can be fastened to each, other or to the outer side 4 of the base body 2 of the support bandage 1. In the middle, in a non-elastic and flexible strap segment 15, a fixing means 16 is likewise arranged which, for example, can be designed as a hook or loop area of a velcro-type fastener. The strap ends with the fixing means 16 are likewise adjoined by non-elastic strap segments 15, Between the non-elastic strap segments 15, elastic and non-elastic sections 13, 12 are arranged and connected in parallel. In the illustrative embodiment shown, the non-elastic section 12 is shorter than the elastic section 13 in the unstretched state. The supporting or fixing strap 10 according to FIG. 5 is thus present at the maximum extended length and has tab-like elastic sections 13. When the supporting or fixing strap 10 according to FIG. 5 is fitted in place, it is initially rigid. The supporting or fixing strap 10 can also be applied directly to a body part, for example in order to stabilize and immobilize a joint. It is likewise possible to apply this supporting or fixing strap 10 to a prosthesis or orthosis or, as has been described, such that it supports a cuff or bandage. For this purpose, the supporting or fixing strap 10 can also be applied to the respective orthopedic device via the fixing means 16 and secured thereon.

After the joint or the orthopedic device has been stabilized, it is possible, by cutting through the non-elastic sections 12 or one non-elastic section 12, to make available a certain degree of elasticity, made available by the elastic sections 13 or by the released elastic section 13 made active. In the illustrative embodiment shown, two areas with parallel elastic and non-elastic sections 13, 12 are provided, but it is also possible for more than two such sections to be arranged in one strap 10.

A variant of the supporting strap 10 is shown in FIG. 6, in which the same reference signs designate the same components. Unlike the situation in FIG. 5, the elastic section 13 in the unstretched state is shorter than the non-elastic section 12, such, that a certain degree of elasticity is present in the supporting and. fixing strap 10 right from the outset. The length of the non-elastic section 12, which in the non-elastic state shown is longer than the length of the unstretched elastic section 13, is preferably chosen such that the elastic section 13 is not overstretched or cannot be extended beyond the intended extension range. Excess loading of the elastic area 13 is thereby avoided, as a result of which a longer service life is ensured, since the elastic section 13 is not extended beyond the maximum extension limit.

In addition to the illustrative embodiments shown, it is possible to combine the different lengths of the elastic and non-elastic sections 12, 13, such that one or more parallel elastic and non-elastic sections 12, 13 according to FIG. 5 can be combined, with the elastic and non-elastic sections 12, 13 according to FIG. 6. It is thus possible, by cutting through the “short” non-elastic sections 12, to make available an increased elasticity of the supporting or fixing strap 10 if the latter already has a basic elasticity safeguarded by the non-elastic sections 12.

A particular design of the supporting strap is shown in FIG. 7, in which a plurality of elastic strap sections 13, in the present, case three elastic strap sections, can be arranged and connected parallel to one another. With this plurality of elastic sections 13, it is possible to provide greater elastic resistance. By-removal, cutting through or the like, it is possible to adjust this elastic resistance.

The elastic sections 13 can be sewn, adhesively bonded or welded onto the strap material. The non-elastic sections 12 can be made of the same material as the non-elastic strap segments 15. A continuous, one-piece strap composed of non-elastic sections 12 and non-elastic strap segments 15 is then formed. It is also possible for the fixing means 16 to be sewn on, adhesively bonded on or welded on. Alternatively, these fixing means 16 can be sewn on at ends of the strap 1, such that they are attached to the respective ends of the non-elastic strap sections 12 or non-elastic strap segments 15.

FIG. 8 shows a variant of the invention corresponding substantially to the embodiment according to FIG. 3. Instead of a multi-part design of the supporting strap 10, which in FIG. 3 has a non-elastic section 12 and an elastic section 13, the supporting strap 10 according to FIG. 8 is one elastic section, such that the entire supporting strap 10 is elastically deformable. As in the previously described embodiments, the elastic supporting straps 10 can be arranged at areas with form-fitting elements, and these areas can also be arranged reversibly on the outer side 4 of the base body 2. The inner side of the base body 2 can be coated at certain points or across the entire surface area, for example with a polyurethane, a silicone or a similar anti-slip material. As also in the previously described embodiments, the base body 2 can be made of a material which possesses shear stability and which does not permit a shifting of the top face relative to the underside or of the outer side relative to the inner side or permits this only to an inappreciable extent.

FIG. 9 illustrates a variant of the invention according to FIG. 1 and shows that a knee bandage 1 with a hose-like base body 2, on which areas 6 with form-fitting elements are arranged for fastening the supporting straps 10, which are guided crosswise here. The areas 6 are designed, for example, as loop-covered areas, which are part of a velcro-type fastener in interaction with hook elements on the tensioning straps 10. The tensioning straps 10 in the present illustrative embodiment are likewise designed as purely elastic tensioning straps 10 which, for example, can be made of an elastic plastic, a rubber material, an elastic fabric or the like. The areas 6 can be secured via velcro-type fasteners to physiologically suitable locations on the base body 2. The material of the base body 2 is very thin and has a thickness of less than one millimeter, resulting in a high degree of elasticity. Because of the short distance of the supporting straps 10 from the skin surface, the tensile forces and pressure forces exerted by the supporting straps 10 are transferred directly to the skin and to the extremity lying below. The supporting straps 10 can be applied with different elasticities, such that a suitably adapted tensile force and tensioning can be exerted on the body part.

FIG. 10 shows a variant of the supporting strap 10 in which, once again, a purely elastic configuration of the supporting strap 10 is provided. At different areas of the supporting strap 10, fixing means 16 are mounted on the underside, for example sewn on, adhesively bonded on or welded on. In the illustrative embodiment shown, the fixing means 16 in the form of hook elements of a velcro-type fastener are arranged at both, ends and at the middle of the supporting strap 10, in order also to ensure an angled arrangement of the supporting strap 10 on the base body.

In addition to the illustrative embodiments shown, the base bodies can also have different configurations, for example designed like pants or a shirt, in order to be able to provide support on the respectively chosen areas of the body.

FIG. 11 shows a detail of a supporting strap 10 that can also be designated as a local compression element, since this supporting strap 10, like the supporting straps in the previous FIGS., exerts a local compression on the underlying muscle tissue, so as to achieve proprioception of the muscle. The ends of the supporting strap 10 are provided with form-fitting elements 16 by way of which the supporting strap 10 can be fastened on a base body. Between the form-fitting elements 16 there are perforations 18, so as to be able to adjust the tensioning elasticity, in particular in the longitudinal extent of the supporting strap 10. Anti-slip coatings, as are shown in FIG. 2 for example, can be applied on the underside of the supporting strap 10. It is thus possible to avoid unwanted sliding when the supporting strap 10 is applied directly to the skin. The form-fitting elements 16 are arranged either on the side of the supporting strap 10 facing away from or facing toward the skin surface, depending on how the supporting strap 10 is secured on the base body. If it is secured purely on the outside, the form-fitting elements, for example in the form of velcro-type fasteners, press buttons or the like, are arranged on the underside, that is to say the side facing toward the body. A similar arrangement is present when the supporting strap 10 is guided on the inside, i.e. areas of the supporting strap bear directly on the skin or are guided in a guide inside the base body 2, and the supporting strap 10 is fastened on the outer side of the base body 2. If complete securing on the inner side of the base body is provided, the form-fitting elements 16 are arranged on that side of the supporting strap 10 facing away from the surface of the body. The orientations relate in each case to a correctly applied support bandage.

FIG. 12 shows an applied support bandage 1 with a base body 2 and supporting straps 10, which lie on the inside and are covered externally by the base body 10. In the illustrative embodiment shown, the supporting straps 10 are guided crossing each other, in order to provide muscle-activating compression in the popliteal area of a knee bandage.

FIG. 13 shows another view of the embodiment according to FIG. 12, namely an oblique plan view from in front. It will be seen that passages 210 in the form of slits are made in the base body 2, through which slits the ends of the supporting straps 10 are guided. The form-fitting elements are arranged on these ends, such that, after the supporting straps 10 have been passed through the passages 210, the supporting straps 10 can be easily fixed on the outer side of the base body 2. If the compression effect decreases, for example as a result of warming during sports activities or as a result of a reduction in circumference caused by detumescence, the tension of the supporting straps 10 can be adjusted by simply releasing the form-fitting elements 16, fastened reversibly on the surface of the base body 2, and tightening the preferably elastic supporting straps 10. It is likewise possible to reduce the compression effect if the tension is felt to be too tight, and this is done by releasing the form-fitting elements 16 from the surface or from the corresponding form-fitting elements of the base body 2 and fastening them back on the base body 2 closer to the passage 210.

A variant of the invention is shown in FIG. 14, in which the supporting straps 10 guided on the inside follow a different course than in FIG. 13. The frontal supporting strap 10 extends substantially parallel to the muscle course, at a slight angle thereto, and the lower supporting strap acts on the patellar tendon, in order to achieve pretensioning.

The support bandages 1 serve in particular for the stimulation of muscles or muscle pretensioning, in order to compensate for possible imbalances, A movement limitation is not generally intended. Particularly in support bandages 1 consisting exclusively of components that are flexible in all directions, a movement limitation is not possible and not intended.

1. A support bandage comprising a base body (2) that is made of an elastic textile material and that has an inner side (3) facing toward the body of a bandage user and an outer side (4) facing away from the body of the bandage user, with an anti-slip coating (5) arranged on the inner side (3), characterized in that the support bandage (1) has at least one supporting strap (10), which is arranged reversibly on the base body (2) by way of form-fitting elements (21).

2. The support bandage as claimed in claim 1, characterized in that the supporting strap (10) has at least one flexible, non-elastic section (12).

3. The support bandage as claimed in claim 1 or 2, characterized in that the supporting strap (10) consists of the non-elastic section (12).

4. The support bandage as claimed in claim 1 or 2, characterized in that the supporting strap (10) has an elastic section (13) arranged parallel to the non-elastic section (12), and the non-elastic section (12) is secured on the elastic section (13).

5. The support bandage as claimed in claim 4, characterized in that the non-elastic section (12) is fixed permanently on the elastic section (13) and has a length that is different than the length of the unstretched elastic section (13).

6. The support bandage as claimed in claim 4, characterized in that the non-elastic section (12) is fastened reversibly on the elastic section (13).

7. The support bandage as claimed in claim 6, characterized in that the non-elastic section (12) is fastened on the elastic section (13) by way of form-fitting elements (23).

8. The support bandage as claimed in one of claims 4 through 7, characterized in that the elastic and non-elastic sections (12, 13) are arranged over each other and at least partially cover each other.

9. The support bandage as claimed in one of claims 4 through 8, characterized in that several elastic and non-elastic sections (12, 13) arranged parallel to one another are arranged in series,

10. The support bandage as claimed. in claim 9, characterized in that a non-elastic strap segment (15) is arranged between two elastic sections (13).

11. The support bandage as claimed in one of claims 4 through 10, characterized in that the non-elastic section (12) is shorter than the unstretched elastic section (13).

12. The support bandage as claimed in one of claims 4 through 10, characterized in that the non-elastic section (12) is longer than the unstretched elastic section (13).

13. The support bandage as claimed in one of claims 4 through 12, characterized in that several elastic sections (13) are arranged parallel to one another.

14. The support bandage as claimed in one of the preceding claims, characterized in that fixing means (16) on the supporting strap (10) are designed as hook and loop areas.

15. The support bandage as claimed in claim 14, characterized in that the fixing means (16) are arranged on the non-elastic sections (12) or on non-elastic strap segments (15).

16. The support bandage as claimed in one of the preceding claims, characterized in that the base body (2) is designed as a sleeve.

17. The support bandage as claimed in one of the preceding claims, characterized in that the anti-slip coating (5) is arranged in areas on the inner side (3) of the base body (2).

18. The support bandage as claimed in one of the preceding claims, characterized in that areas (6) with form-fitting elements (21) for fastening the supporting strap (10) are arranged on the outer side (4) of the base body (2).

19. The support bandage as claimed in one of the preceding claims, characterized in that the form-fitting elements (21) are fastened reversibly on the base body (2).

20. The support bandage as claimed in one of the preceding claims, characterized in that the outer side (4) of the base body (2) is designed as a loop-covered layer, and the form-fitting elements (16) of the supporting strap (10) are designed as hook elements.

21. The support bandage as claimed in one of the preceding claims, characterized in that the base body (2) has at least one recess (7).

22. The support bandage as claimed in one of the preceding claims, characterized in that the supporting strap (10) has a force indicator (17).

23. The support bandage as claimed in one of the preceding claims, characterized in that a guide (8) for the supporting strap (10) is worked into the base body (2).

24. The support bandage as claimed in one of the preceding claims, characterized in that the supporting strap (10) has a curved basic shape.

25. The support bandage as claimed in one of the preceding claims, characterized in that elastic elements are secured on, or perforations are introduced into, the textile material of the base body (2) or the supporting strap (10) and modify the elasticity of the support bandage in some areas .

26. The support bandage as claimed in one of the preceding claims, characterized in that the material of the base body (2) possesses shear stability.

27. The support bandage as claimed in one of the preceding claims, characterized in that the supporting strap (10) is arranged at least partially on the inner side of the base body (2).

28. The support bandage as claimed in one of the preceding claims, characterized in that, on the inner side facing toward the skin surface, the supporting strap (10) has an anti-slip coating (5).

29. The support bandage as claimed in one of the preceding claims, characterized in that the base body (2) has passages (210) for the supporting strap (10).
